(19) United States
(12) Patent Application Publication
(10) Pub. No.: US 2005/0283087 A1
(43) Pub. Date: Dec. 22, 2005

(54) Device and method for central blood pressure estimation

(75) Inventors: Kenji Takazawa, Tsurugashima-shi (JP); Hironori Satoh, Moriyama-shi (JP); Yoshinori Miyawaki, Otsu-shi (JP); Tatsuya Kobayashi, Otsu-shi (JP); Shogo Sawada, Kyoto-shi (JP)

Correspondence Address: MORRISON & FOERSTER LLP, 1650 TYSONS BOULEVARD, SUITE 300, MCLEAN, VA, 22102, US

(73) Assignee: OMRON Healthcare Co., Ltd., Kyoto-shi (JP)

(21) Appl. No.: 11/151,648

(22) Filed: Jun. 14, 2005

(30) Foreign Application Priority Data
Jun. 15, 2004 (JP) 2004-176906

(51) Int. Cl. A61B 5/02

(52) U.S. Cl. 600/500; 600/503

(57) ABSTRACT

A sensor unit is brought into contact with a surface of a living body to detect a pulse wave of an artery directly below. A CPU extracts a characteristic point of the pulse wave detected and obtains a second systolic component in the pulse wave using the characteristic point extracted. Then, a variation with time in a systolic blood pressure of a central artery of the living body is estimated using the second systolic component obtained, and an estimated variation with time in the systolic blood pressure is displayed on a display portion. For estimation of the systolic blood pressure, arithmetic with a linear transformation is applied using the second systolic component obtained and a blood pressure of a peripheral artery which is measured beforehand with an electronic sphygmomanometer.

### BACKGROUND OF THE INVENTION

1. Field of the Invention

The present invention relates to a device and a method for central blood pressure estimation. More specifically, the present invention relates to a device and a method for central blood pressure estimation with which a variation in a systolic blood pressure of a central artery can be estimated.

2. Description of the Background Art

When a circulation in a coronary artery falls into a bad condition and an acute heart disease such as myocardial infarction occurs, for example, a coronary vasodilator such as nitroglycerine is administered to a patient. An effect of such drug administration can be ensured when a blood pressure of a central artery is decreased due to the drug administration. For accurate measurement of the blood pressure of the central artery, a doctor conventionally performs invasive measurement with inserting a catheter into a body of a patient, which may give pain to the patient and is not practical because an apparatus of a large scale is required. Therefore, monitoring of a variation in a blood pressure of a central artery through a peripheral artery has been desired as a manner to easily monitor a variation in the blood pressure of the central artery without giving a pain.

A peripheral blood pressure can be easily measured non-invasively using a commercially available electronic sphygmomanometer. It is known, however, that an internal pressure of an aorta extending from a heart to a brain and a kidney (a central blood pressure) is often different from a blood pressure measured for a relatively thin peripheral blood vessel at a brachium or the like (a peripheral blood pressure). As described in Japanese Patent Laying-Open No. 07-039530, for example, it is known that the central blood pressure varies even when the peripheral blood pressure does not vary. Therefore, a variation in the central blood pressure cannot be always recognized accurately by monitoring a variation in the peripheral blood pressure with the electronic sphygmomanometer.

In Japanese Patent Laying-Open No. 2003-000555 or 2002-051995, a technique is proposed to more accurately estimate a pulse waveform of a central artery, that is, a central blood pressure waveform.

Since a transfer function is used in the technique proposed in Japanese Patent Laying-Open No. 2003-000555, a complicated operation such as a Fourier transform is required, and therefore a device including a processor having a function of high-speed operation is required, which results in an increased price of the device. In addition, Japanese Patent Laying-Open No. 2002-051995 uses a manner in which a variation in a pressure pulse wave (a time-varying blood vessel-conducted wave generated by a variation in an internal pressure of an artery is referred to as a pressure pulse wave) during a propagation thereof from a central artery to a peripheral artery is derived from an artery model and a pulse wave propagation speed, and a central blood pressure is measured from a measured peripheral blood pressure, which requires a long operation time before an analysis without use of a high-performance operation device, and is not practical because a long time is required to recognize a phenomenon of the central blood pressure after an occurrence thereof.

In addition, though Japanese Patent Laying-Open No. 07-039530 suggests that a second systolic component decreases after drop of a central pressure, it does not refer to an increase thereof or the like.

### SUMMARY OF THE INVENTION

An object of the present invention is to provide a device and a method for central blood pressure estimation with which a variation in a systolic blood pressure of a central artery can be estimated readily.

To attain the object, a central blood pressure estimation device according to an aspect of the present invention includes a pulse wave detection portion brought into contact with a surface of a living body to detect a pulse wave of an artery directly below, a portion for obtaining a second systolic component in the pulse wave detected by the pulse wave detection portion, an estimation portion for estimating a variation with time in a systolic blood pressure of a central artery of the living body using the second systolic component obtained, and a display portion for displaying the variation with time in the systolic blood pressure estimated.

The estimation portion preferably estimates a systolic blood pressure of a central artery of the living body using a blood pressure of the living body measured beforehand and the second systolic component obtained.

The blood pressure of the living body measured beforehand is preferably a blood pressure of a peripheral artery of the living body.

Preferably, a linear transformation is used in the estimation portion.

The display portion preferably successively displays the systolic blood pressure estimated by the estimation portion in time sequence.

Preferably, the systolic blood pressure for each heart beat is successively displayed.

The display portion preferably displays the systolic blood pressure or a variation thereof with time associated with another index relating to a blood pressure.

The index is preferably a blood pressure of a peripheral artery of the living body or a variation thereof with time.

The display portion preferably displays the systolic blood pressure or a variation thereof with time and the blood pressure of the peripheral artery of the living body or a variation thereof with time in comparison with each other on a two-dimensional graph.

A central blood pressure estimation device according to another aspect of the present invention includes a pulse wave detection portion brought into contact with a surface of a living body to detect a pulse wave of an artery directly below, a portion for obtaining a second systolic component in the pulse wave detected, a calculation portion for calculating a variation with time in the second systolic component obtained, and a display portion for displaying the variation with time in the second systolic component calculated.

The second systolic component is a reflected wave component which is one of factors determining a systolic blood pressure in a central artery.

To attain the object described above, a method for estimating a central blood pressure according to a further aspect of the present invention includes the steps of detecting a pulse wave using a sensor brought into contact with a surface of a living body to detect a pulse wave of an artery directly below, obtaining a second systolic component in the pulse wave detected in the step of detecting a pulse wave, estimating a systolic blood pressure of a central artery of the living body using the second systolic component obtained, and displaying a variation with time in the systolic blood pressure estimated in the step of estimating.

According to the present invention, the second systolic component of the pulse wave detected from a living body can be obtained, which second systolic component (reflected wave) is one of the factors determining a systolic blood pressure in a central artery. With this, a variation with time in the systolic blood pressure in the central artery can be estimated using the second systolic component. As a result, the variation with time in the systolic blood pressure in the central artery can be estimated readily within a short time.

In addition, a doctor can monitor a state of a heart, that is, a central artery of a living body referring to a display of the variation with time in the systolic blood pressure of the central artery.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 shows a way of connection between a sensor unit and a fixing base in an embodiment of the present invention.

FIG. 2 shows a way of using for pulse wave measurement in the embodiment of the present invention.

FIGS. 3A to 3E show a construction of the sensor unit in the embodiment of the present invention.

FIG. 4 shows a functional construction of a device in the embodiment of the present invention.

FIG. 5 is a flow chart of processing for central blood pressure estimation in the embodiment of the present invention.

FIG. 6 is a diagram for describing a filter switching circuit forming a characteristic variable filter in the embodiment of the present invention.

FIG. 7A shows a frequency characteristic of a filter A, and FIG. 7B shows a frequency characteristic of a filter B.

FIG. 8 shows an example of mode transition of the characteristic variable filter in the embodiment of the present invention.

FIG. 9 is a flow chart of processing procedures for reflected wave calculation and central arterial pressure calculation with a sensor signal analysis in the embodiment of the present invention.

FIG. 10A shows an example of display of a pulse wave for one pulse when a frequency characteristic of the characteristic variable filter is set to a characteristic A, and FIG. 10B shows an example of display of a pulse wave for one pulse when a frequency characteristic of the characteristic variable filter is set to a characteristic B.

FIG. 11 shows a modified example of the characteristic variable filter in the embodiment of the present invention.

FIG. 12 is a diagram for describing a procedure for the central arterial pressure calculation in the embodiment of the present invention.

FIG. 13 is a diagram for describing a procedure for the central arterial pressure calculation in the embodiment of the present invention.

FIG. 14 is a trend graph indicating variations in a systolic blood pressure with time, which are obtained from concurrently performed invasive measurement with a catheter and estimation according to the embodiment.

FIG. 15 shows an example of a display screen in the embodiment of the present invention.

FIG. 16 shows another example of the display screen in the embodiment of the present invention.

FIGS. 17A and 17B respectively show variations in a pressure pulse wave of a peripheral artery and a pressure pulse wave of a central artery, which were concurrently measured for the same subject.

FIGS. 18A and 18B are graphs indicating a result of measurement of central arterial pressures before and after a drug administration with insertion of a catheter.

FIGS. 19A and 19B show reduction of a reflected wave in a pressure pulse wave of a peripheral artery due to the drug administration.

FIG. 20 is a graph showing a reflected wave trend of a pressure pulse wave of a peripheral artery and a result of measurement in an experiment of a central arterial pressure.

### DESCRIPTION OF THE PREFERRED EMBODIMENT

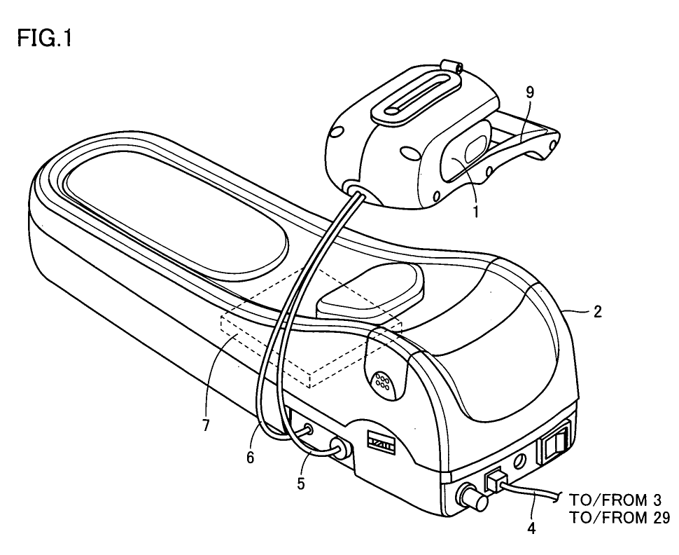

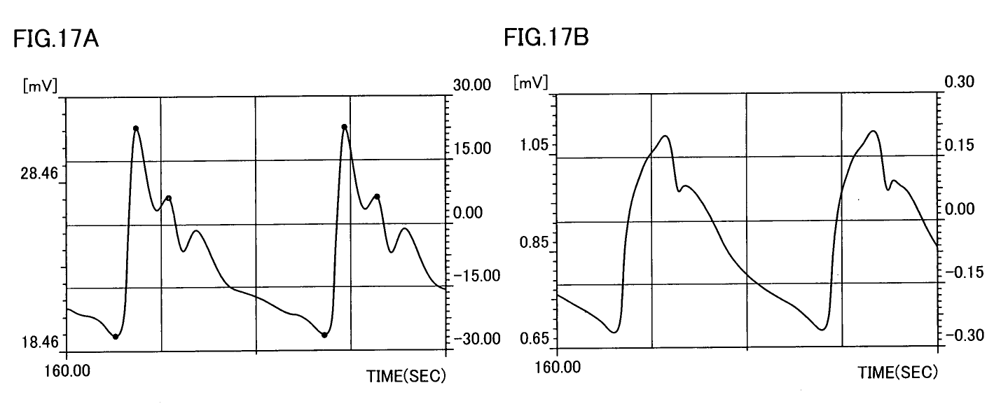

An embodiment of the present invention will now be described referring to the drawings. It is to be noted that, the same or corresponding portions in the drawings are indicated with the same characters, and descriptions thereof will not be repeated. FIGS. 1-16 are diagrams for describing a device or a method according to this embodiment, and FIGS. 17A and 17B-20 are diagrams for describing a technical principle according to this embodiment.

<Description of Principle for Embodiment>

FIGS. 17A and 17B respectively show variations in a pressure pulse wave of a peripheral artery and a pressure pulse wave of a central artery, which were concurrently measured for the same subject. Waveforms of FIGS. 17A and 17B depend on a state of the central artery. A waveform of the central artery shown in FIG. 17B is measured invasively with inserting a catheter, and a pressure pulse wave of the peripheral artery shown in FIG. 17A is obtained by measuring with oscillometry a blood pressure of a brachium using an electronic sphygmomanometer or the like. An axis of abscissas in each of FIGS. 17A and 17B indicates a lapse of time, while an axis of ordinates indicates a voltage level (mV) of a pressure pulse wave signal (a voltage signal), which voltage level is proportional to a blood pressure.

The pressure pulse wave of the central artery shown in FIG. 17B has a large amplitude level of a reflected wave in many cases, and a maximum blood pressure (a systolic blood pressure) of the central artery depends on the amplitude level of the reflected wave if a subject has such a large amplitude level of the reflected wave. FIG. 17A shows a variation in a pressure pulse wave of a peripheral artery. Sometimes, a traveling wave is large in the pressure pulse wave of the peripheral artery of the subject who has the large amplitude level of the reflected wave of the pressure pulse wave in the central artery. This results from a transfer characteristic of the pressure pulse wave such that, a component of a certain frequency (about 5 Hz) is emphasized during transmission through a blood vessel. In this situation, an amplitude of an ejection wave when blood is sent from a heart with a heart beat often becomes large, and a maximum blood pressure (a systolic blood pressure) of the peripheral artery largely depends on an amplitude level of the ejection wave.

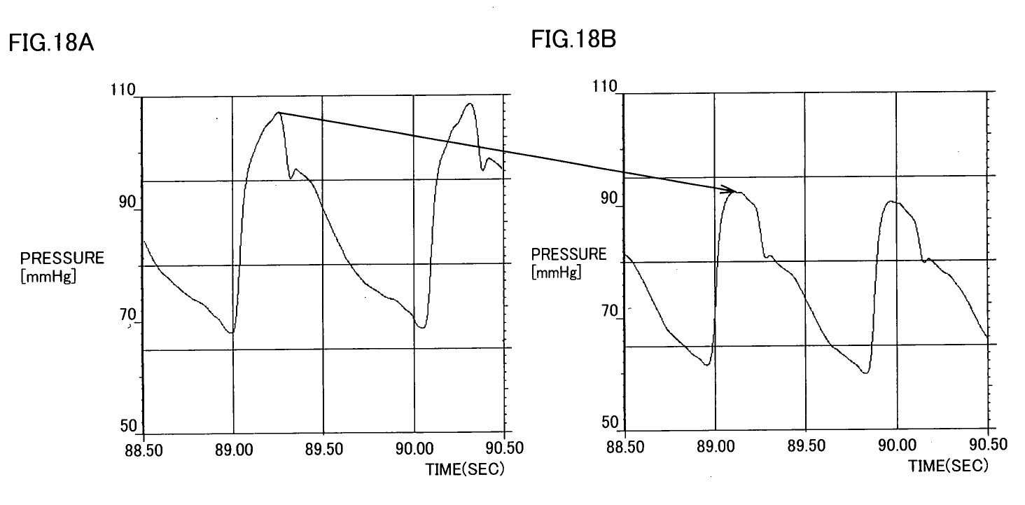

FIGS. 18A and 18B are graphs indicating a result of measurement of blood pressures of a central artery before and after an administration of nitroglycerine, which measurement was performed with insertion of a catheter into a body of a subject. An axis of abscissas in each graph indicates a lapse of time, while an axis of ordinates indicates an amplitude level of a pressure pulse wave in a unit of a pressure (mmHg). FIG. 18A, before a nitroglycerine administration, and FIG. 18B, after the nitroglycerine administration, show that a level of a peak of an amplitude of the pressure pulse wave is decreased as indicated with an arrow in the drawings, which means that a reflected wave component in the pressure pulse wave of the central artery is decreased resulting from an action of nitroglycerine to blood vessel cells to dilate the blood vessel.

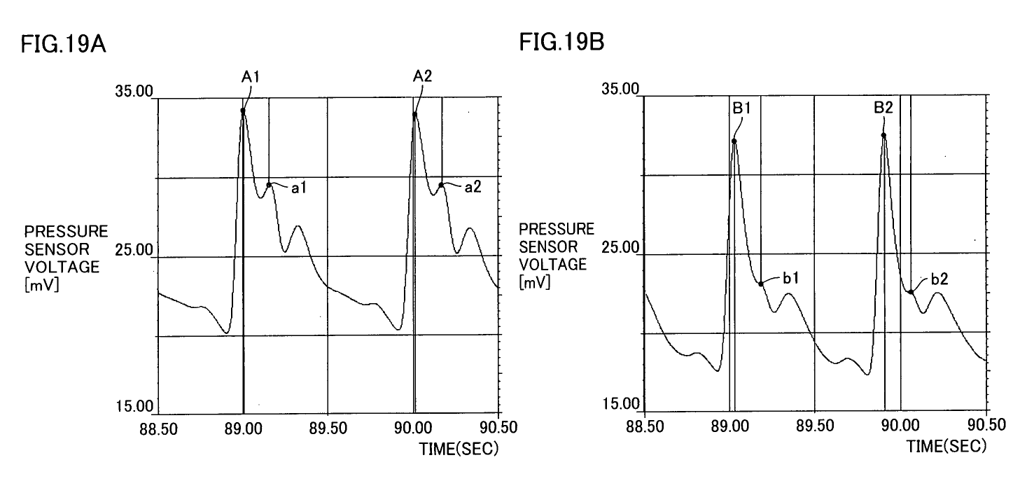

FIGS. 19A and 19B are graphs showing reduction of the reflected wave in the pressure pulse wave of the peripheral artery due to the administration of nitroglycerine. An axis of abscissas in each graph indicates a lapse of time, while an axis of ordinates indicates an amplitude level of the pressure pulse wave in a unit of a voltage (mV). The pressure pulse wave of the peripheral artery shown corresponds to a waveform of a blood pressure measured with oscillometry using the electronic sphygmomanometer. In the waveform of the pressure pulse wave of the peripheral artery, blood pressures are indicated at points “A1”, “A2”, “B1”, and “B2”, each is a peak of an amplitude of the pressure pulse wave. As described in Japanese Patent Laying-Open No. 07-39530, the reflected wave component which appears in the pressure pulse wave of the central artery appears at each of points “a1”, “a2”, “b1”, and “b2” in the pressure pulse wave of the peripheral artery. Therefore, it is apparent that an amplitude level of the pressure pulse wave indicating the blood pressure in the peripheral artery is not affected by the reflected wave. As shown in the drawings, since the blood vessel is dilated due to the nitroglycerine administration, the amplitude level of the reflected wave appearing in FIG. 19B is reduced as compared with that shown in FIG. 19A and, as a result, it is recognized that a level of point “a1” is decreased to a level of point “b1” while a level of point “a2” is similarly decreased to a level of point “b2”.

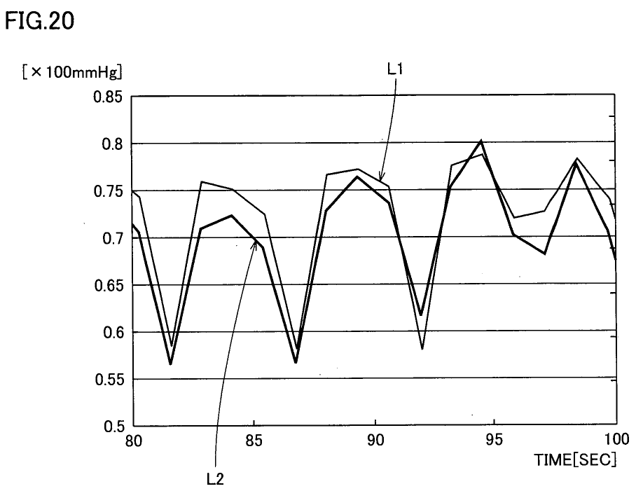

FIG. 20 shows a graph L1 indicating a trend in the amplitude level of the reflected wave (hereafter referred to as a reflected wave trend) in the pressure pulse wave of the peripheral artery and a central arterial pressure trend L2 indicating a trend in a variation in the central arterial pressure (a maximum value: a systolic blood pressure), which use actually measured values resulting from an experiment. In the graph shown in FIG. 20, an axis of abscissas indicates a lapse of time, and an axis of ordinates indicates peak values of pressure pulse waves of the central arterial pressure (the systolic blood pressure) and a peripheral arterial pressure (both represented in a unit (×100 mmHg)). As described above, since the central arterial pressure, that is, the systolic blood pressure of the central artery depends on the amplitude level of the reflected wave, the systolic blood pressure increases when the amplitude level of the reflected wave increases, and the systolic blood pressure decreases when the amplitude level decreases. In contrast, in the pressure pulse wave of the peripheral artery, an effect of the reflected wave appears at points “a1”, “a2”, “b1”, and “b2” in FIGS. 19A and 19B as second systolic components of systolic blood pressures (blood pressures at points “A1”, “A2”, “B1”, and “B2” in FIGS. 19A and 19B).

Therefore, as shown in FIG. 20, a turning point of a reflected wave trend L1 appears slightly after a turning point of central arterial pressure trend L2. In FIG. 20, the turning points are shown in association with each other with a slight time difference. The turning point appears for every one pulse of a heart beat, and reflected wave trend L1 is obtained by plotting of a peak value of the reflected wave for each heart beat as the turning point in time sequence. Since it is apparent from the graph in FIG. 20 that the variation in the amplitude level of the reflected wave in the peripheral artery nearly corresponds to the variation in the systolic blood pressure in the central artery, it can be said as a result of this experiment that a variation with time (a trend) in time sequence of the systolic blood pressure in the central artery can be recognized readily by monitoring reflected wave trend L1 in the pressure pulse wave of the peripheral artery.

<Exterior and Construction of Device According to this Embodiment>

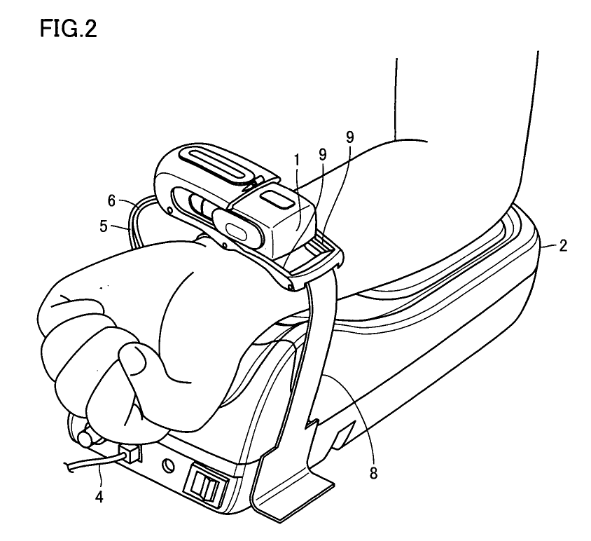

FIG. 1 shows a connection between a sensor unit and a fixing base. FIG. 2 shows a state of a pulse wave detection device applied to a central blood pressure estimation device according to this embodiment, which is mounted on a living body.

Referring to FIGS. 1 and 2, the pulse wave detection device includes a sensor unit 1 which is mounted on a surface of a wrist to detect a pulse wave of a radial artery of the wrist, a fixing base 2 for fixing the wrist for pulse wave detection, and a display unit 3 (not shown) for inputting and outputting various information regarding the pulse wave detection. In FIG. 1, sensor unit 1 is located inside a housing. In FIG. 2, sensor unit 1 is slid via a slide groove 9 (see FIG. 1) to move outside the housing and is located on the wrist.

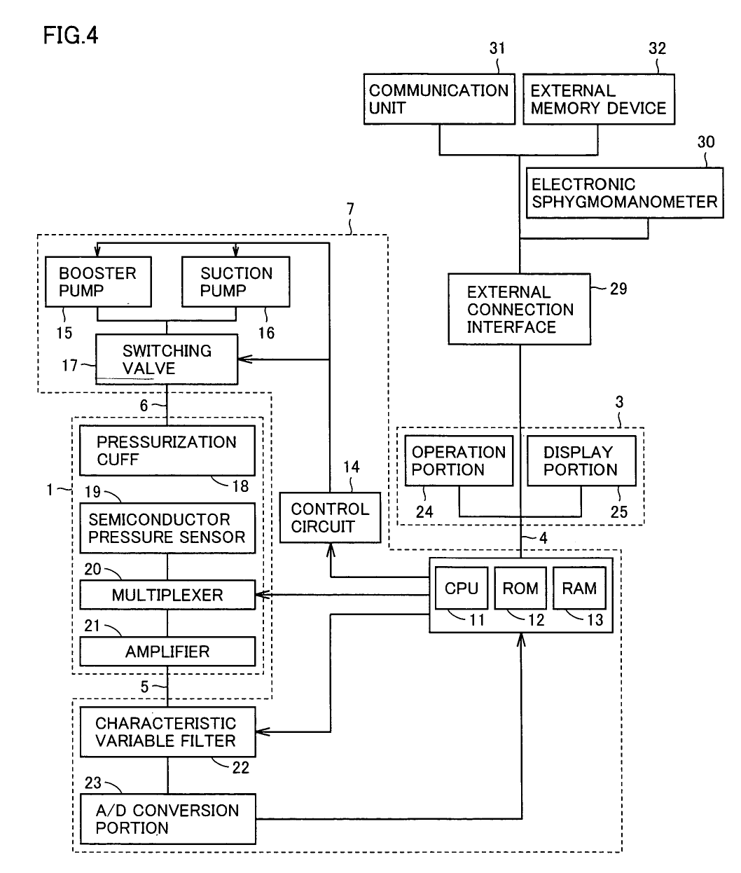

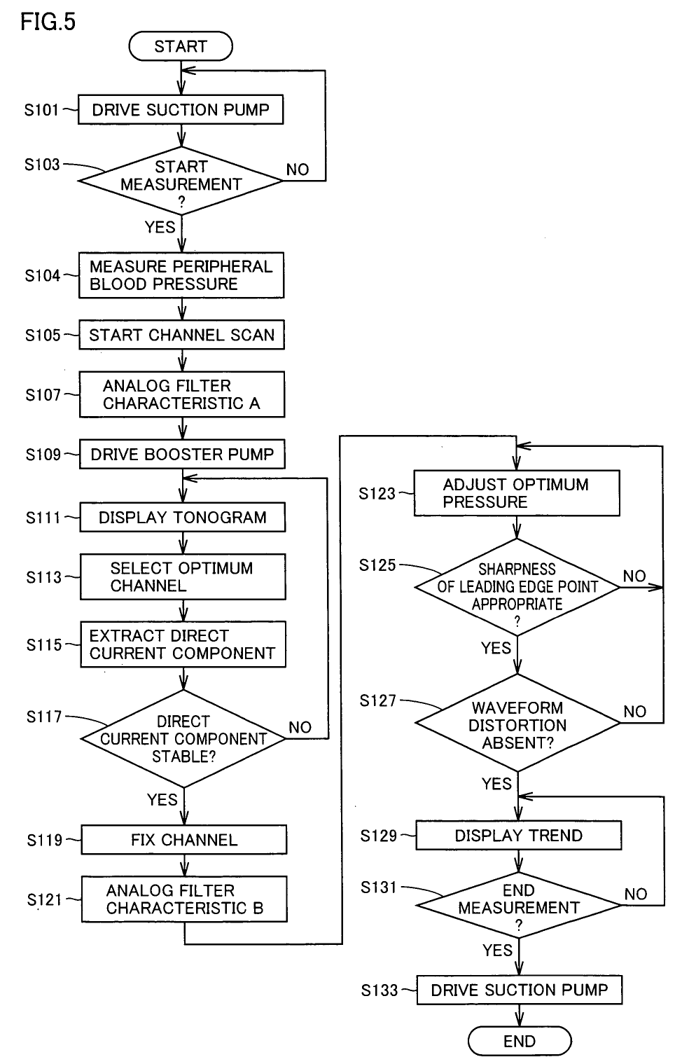

Fixing base 2 has a fixing base unit 7 provided therein. Fixing base unit 7 is connected to display unit 3 and an external connection interface 29 shown in FIG. 4 described below via a USB (Universal Serial Bus) cable 4 to allow communication therewith. In addition, fixing base unit 7 and sensor unit 1 are connected via a communication cable 5 and an air tube 6.

As shown in FIG. 2, to detect a pulse wave, a user mounts a wrist on a prescribed position on fixing base 2, slides sensor unit 1 to locate it on a surface of an artery side of the wrist, and fastens the housing of sensor unit 1 and fixing base 2 with a belt 8 to immobilize sensor unit 1 on the wrist.

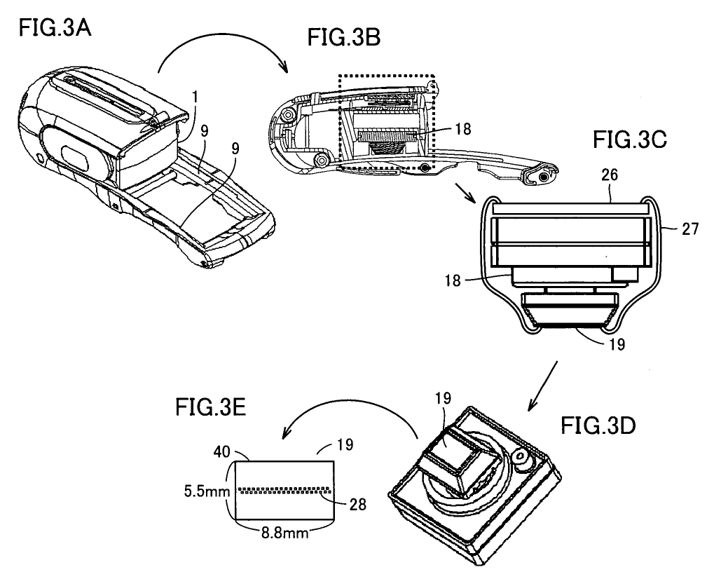

FIGS. 3A to 3E show a construction of sensor unit 1. FIG. 3B shows a cross-sectional structure of sensor unit 1 shown in FIG. 3A, which is cut in a direction across the wrist on which the sensor unit is mounted. A portion inside a box indicated with broken lines in FIG. 3B is enlarged and shown in FIG. 3C. A cuff pressure of a pressurization cuff 18 shown in FIG. 3B is adjusted with a booster pump 15 and a suction pump 16 described below, and a semiconductor pressure sensor 19 attached via a block molded with ceramic or resin freely moves upward or downward with an amount corresponding to a level of the cuff pressure. Semiconductor pressure sensor 19 is moved downward to project from an opening previously provided in the housing, and is pressed against a surface of the wrist.

As shown in FIGS. 3D and 3E, an arrangement of a plurality of sensor elements 28 in semiconductor pressure sensor 19 extends in a direction corresponding to a substantially perpendicular (crossing) direction to an artery when sensor unit 1 is mounted on the wrist, and has a length longer than a diameter of the artery. When pressed with a cuff pressure of pressurization cuff 18, each of sensor elements 28 outputs pressure information, which is a pressure oscillation wave generated from the artery and transmitted to a surface of a living body, as a voltage signal (hereafter referred to as a “pressure signal”). In this embodiment, 40 sensor elements 28, for example, are arranged on a measurement surface 40 having a prescribed dimension (5.5 mm×8.8 mm).

Referring to FIG. 3C, the pressure signal from sensor element 28 is sent via a flexible wiring 27 to a multiplexer 20 and an amplifier 21 shown in FIG. 4 which are in a PCB (Printed Circuit Board) 26, successively.

FIG. 4 shows a functional construction of the central blood pressure estimation device according to the embodiment of the present invention. Referring to FIG. 4, display unit 3 includes an operation portion 24 provided to allow operation from the outside and operated to input various information including information regarding pulse wave detection, and a display portion 25 formed with an LED (Light Emitting Diode), an LCD (Liquid Crystal Display) or the like for outputting various information such as results of artery position detection, pulse wave measurement and central blood pressure estimation to the outside.

Fixing base unit 7 includes an ROM (Read Only Memory) 12 and an RAM (Random Access Memory) 13 for storing data or a program to control operations for the pulse wave detection and central blood pressure estimation, a CPU (Central Processing Unit) 11 for performing various processing including computing for concentrated control of operations of the device, booster pump 15, suction pump 16, a switching valve 17, a control circuit 14 for transmitting a signal received from CPU 11 to booster pump 15, suction pump 16 and switching valve 17, a characteristic variable filter 22 which has at least two characteristic values and can be varied to any of the characteristic values, and an A/D conversion portion 23. An electronic sphygmomanometer 30, a communication unit 31 and an external memory device 32 are connected to CPU 11 via external connection interface 29. Electronic sphygmomanometer 30 measures a blood pressure (a systolic blood pressure and a diastolic blood pressure) of a peripheral artery in a region such as a brachium of a subject using oscillometry in a generally well-known procedure, and outputs data of a measurement result to CPU 11 via external connection interface 29. CPU 11 displays input data of blood pressure measurement result on display portion 25, and refers to the data for central blood pressure estimation.

Though a detection region of a pressure pulse wave (also referred shortly as a pulse wave) of the peripheral artery is a wrist in FIG. 2 and a brachium is made to be a blood pressure measurement region in this embodiment, measurement regions for the pulse wave and blood pressure are not limited to these regions. To obtain higher accuracy of estimation, it is desirable that the measurement regions be in proximity to each other.

In addition, though sensor unit 1 for pulse wave detection and electronic sphygmomanometer 30 for blood pressure measurement are provided separately, the device may have a construction in which both of them are included in the same housing. In this situation, the measurement regions can be readily arranged in proximity to each other as described above.

CPU 11 accesses ROM 12 to read a program and expands the program on RAM 13 for execution to control a whole of the device. CPU 11 receives from operation portion 24 an operation signal from a user and performs control processing of the whole device based on the operation signal. That is, CPU 11 sends a control signal based on the operation signal input from operation portion 24. CPU 11 also displays a result of pulse wave detection and the like on display portion 25.

Booster pump 15 is a pump for boosting an internal pressure (hereafter referred to as a “cuff pressure”) of pressurization cuff (an air bag) 18 described below, and suction pump 16 is a pump for decreasing the cuff pressure. Switching valve 17 selectively switches to one of booster pump 15 and suction pump 16 and connects to air tube 6. Control circuit 14 controls these elements.

Sensor unit 1 includes semiconductor pressure sensor 19 including a plurality of sensor elements 28, multiplexer 20 selectively deriving a pressure signal output from each of the plurality of sensor elements 28, amplifier 21 for amplifying the pressure signal output from multiplexer 20, and pressurization cuff 18 including the air bag having a pressure adjusted to press semiconductor pressure sensor 19 against a wrist.

Semiconductor pressure sensor 19 is formed with a semiconductor chip made of single crystal silicon or the like including the plurality of sensor elements 28 arranged in one direction with a prescribed spacing (see FIG. 3E), and is pressed against a measurement region such as a wrist of a measured subject with a pressure of pressurization cuff 18. In this state, semiconductor pressure sensor 19 detects a pulse wave of the subject via a radial artery. Semiconductor pressure sensor 19 inputs the pressure signal output with detection of the pulse wave to multiplexer 20 for each channel of sensor element 28.

Multiplexer 20 receives and selectively outputs the pressure signal output from each sensor element 28. The pressure signal sent from multiplexer 20 is amplified in amplifier 21 and selectively output to A/D conversion portion 23 via characteristic variable filter 22. In this embodiment, multiplexer 20 is dynamically controlled by CPU 11.

Characteristic variable filter 22 is a low pass filter having a variable cutoff frequency to cut off a signal component of at least a prescribed frequency. Characteristic variable filter 22 will be described below in detail.

A/D conversion portion 23 receives the pressure signal, which is an analog signal derived from semiconductor pressure sensor 19, converts it into digital information, and provides the result to CPU 11. CPU 11 concurrently obtains the pressure signal output from each sensor element 28 included in semiconductor pressure sensor 19 along a time axis via multiplexer 20.

Since CPU 11, ROM 12 and RAM 13 are included in fixing base unit 7 in this embodiment, display unit 3 can be made smaller.

It is to be noted that, though fixing base unit 7 of fixing base 2 and display unit 3 are separately provided, fixing base 2 may include both functions. In addition, though CPU 11, ROM 12 and RAM 13 are included in fixing base unit 7, they may be included in display unit 3. Furthermore, fixing base unit 7 may be connected to a PC (Personal Computer) to perform various control with the PC.

<Operation and Construction of Device in Embodiment of the Present Invention>

An operation of the pulse wave detection device having characteristic variable filter 22 in the embodiment of the present invention will now be described.

FIG. 5 is a flow chart of central blood pressure estimation processing including pulse wave measurement processing in the embodiment. Processing shown in the flow chart of FIG. 5 is performed by CPU 111 which accesses ROM 12 to read a program and expands the program on RAM 13 for execution.

Referring to FIG. 5, when a power supply switch (not shown) is turned on, CPU 11 provides an instruction to control circuit 14 to drive suction pump 16, and control circuit 14 switches switching valve 17 to a side of suction pump 16 based on this instruction and drives suction pump 16 (step (hereafter abbreviated as S) 101). By driving of suction pump 16, the cuff pressure is made sufficiently lower than an atmospheric pressure via switching valve 17, and therefore accidental projection of a sensor portion including semiconductor pressure sensor 19, which causes a malfunction or a failure, can be avoided.

Thereafter, movement of the sensor portion to a measurement region, pressing of a measurement start switch (not shown) included in operation portion 24 or the like is sensed and a determination is made to start measurement (S103). In the former situation, the sensor portion includes a microswitch or the like, which is not shown, for sensing the movement thereof, and CPU 11 determines as to whether the sensor portion has moved or not based on a detection signal of the microswitch.

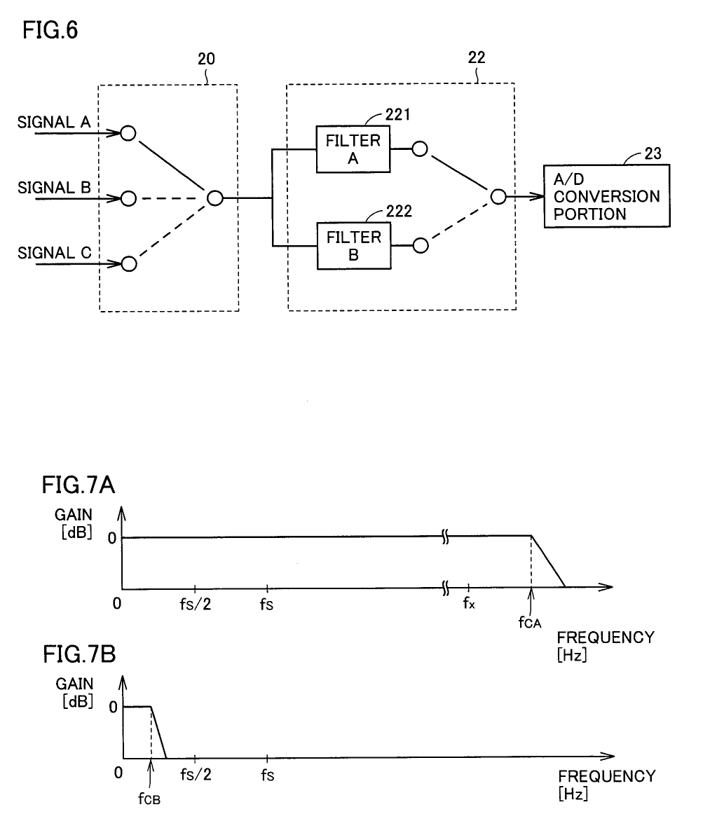

When a start of the measurement is determined (YES in S103), CPU 11 inputs data of a systolic (maximum) blood pressure and a diastolic (minimum) blood pressure of a peripheral artery, which is a result of blood pressure measurement according to oscillometry performed in electronic sphygmomanometer 30 (S104). CPU 11 then operates multiplexer 20 and starts a channel scan to obtain a pressure signal from each sensor element 28 (S105). In this situation, CPU 11 sets a characteristic of a cutoff frequency of characteristic variable filter 22 to a characteristic “A”. As shown in FIG. 6, in this embodiment, a control signal is transmitted to a switching circuit forming characteristic variable filter 22 to select a filter A 221 (hereafter referred to as a filter 221) (S107).

Then, CPU 11 sends a control signal to control circuit 14 to drive booster pump 15. Based on this control signal, control circuit 14 switches switching valve 17 to a side of booster pump 15 and drives booster pump 15 (S109). With this, the cuff pressure is increased and the sensor portion including semiconductor pressure sensor 19 is pressed against a surface of a measurement region of a subject.

When the sensor portion is pressed against the measurement region, the pressure signal from each sensor element 28 included in semiconductor pressure sensor 19 is input with time division via multiplexer 20 and is provided to amplifier 21. Amplifier 21 amplifies the pressure signal provided and outputs the result. An amplified pressure signal is then input to filter 221. The pressure signal filtered with filter 221 is sent to A/D conversion portion 23. A/D conversion portion 23 then converts the provided pressure signal into digital information and outputs the result to CPU 11. CPU 11 inputs the digital information, makes a tonogram using the digital information input, and displays the result on display portion 25 (S111).

Next, CPU 11 detects sensor element 28 located above the artery based on the tonogram made in S111, and executes processing for selecting that sensor element 28 as an optimum channel (S113). It is to be noted that, a technique such as that described in Japanese Patent Laying-Open No. 2004-222847 can be used for the processing for selecting an optimum channel.

In this embodiment, it is assumed that one sensor element 28 is adopted as the optimum channel.

At the same time, CPU 11 extracts a direct current component from the pressure signal based on the digital information of the pressure signal input from each sensor element 28 (S115). The direct current component can be derived from an average value of the pressure signal in a constant time, a component of the pressure signal which passed through the low pass filter (a component after removal of a pulse wave), or a pressure signal level at a leading edge point of a pulse wave (just before mixing of a pulse wave component).

More specifically, in S115, the direct current component can be extracted by dividing an output variation of the pressure signal into windows (sections) each corresponding to a constant time, and calculating an average of output levels in each window. Alternatively, the direct current component can be similarly extracted by, for example, calculating a median value of a maximum value and a minimum value of output levels in each window, or extracting a value of at most a prescribed frequency using a low pass filter. It is to be noted that, the constant time described above is a time interval previously set for pulse wave detection, which is independent of a pulse of a subject, and is preferably about 1.5 seconds which includes a general time for one pulse.

Then, CPU 11 detects a site in the pressure signal input from each sensor element 28 at which the direct current component extracted in S115 is stable (S117). When the site with the stable direct current component is not detected (NO in S117), processing of S111-S117 described above is repeated with continued boosting for pressurization cuff 18 by booster pump 15 until the site with the stable direct current component is detected.

As described above, by concurrently performing processing for selection of the optimum channel and processing for adjustment of an optimum pressure by detection of the direct current component, a time required before a start of pulse wave measurement can be decreased.

It is to be noted that, the optimum pressure may be adjusted after the optimum channel is selected.

When the selection of the optimum channel is completed and the site with the stable direct current component is detected (YES in S117), CPU 11 controls multiplexer 20 to fix the channel. With this, the pressure signal from sensor element 28 determined as the optimum channel is sent continuously (S119). At the same time, CPU 11 switches the characteristic of the cutoff frequency of characteristic variable filter 22 to a characteristic “B” (S121). In this embodiment, a control signal for switching to a filter B 222 (hereafter referred to as a filter 222) shown in FIG. 6 is transmitted to the switching circuit of characteristic variable filter 22.

Then, a pressurization force corresponding to the site with the stable direct current component detected in S117 is determined as an optimum pressurization force of pressurization cuff 18, and a control signal is sent to control circuit 14 to adjust a pressure of pressurization cuff 18 (S123).

After the pressurization force of pressurization cuff 18 is determined as the optimum pressurization force in S123, CPU 11 determines as to whether sharpness of a leading edge point of waveform data, that is, the pressure signal output from sensor element 28 selected as the optimum channel while pressurization cuff 18 is kept with the optimum pressurization force, is appropriate or not (S125), and as to whether there is a waveform distortion or not (S127).

When the sharpness of the leading edge point of the waveform data is inappropriate (NO in S125), or when the waveform distortion is detected (NO in S127), adjustment of the pressurization force in S123 is repeated until the sharpness of the leading edge point of the waveform data becomes appropriate or until the waveform distortion is not detected.

When the sharpness of the leading edge point of the waveform data is appropriate (YES in S125) and the waveform distortion is not detected (YES in S127), CPU 11 obtains the waveform data at that time point via multiplexer 20, amplifier 21, filter 222 and A/D conversion portion 23. The waveform data obtained is successively stored to generate data of reflected wave trend L1 as shown in FIG. 20 based on the stored waveform data, and the reflected wave trend based on generated data is displayed on display portion 25 (S129). A doctor can readily read (estimate) a variation with time in a central arterial pressure referring to reflected wave trend L1 displayed.

In this situation, since the channel is fixed in S119, multiplexer 20 sends only the pressure signal from a single channel to filter 222 via amplifier 21. The pressure signal filtered with filter 222 is then converted into a digital signal in A/D conversion portion 23.

Then, CPU 11 determines as to whether a prescribed condition for ending central blood pressure estimation processing is met or not (S131). The condition for ending central blood pressure estimation processing in S131 may be a lapse of a prescribed time previously set (for example, 30 seconds) from a start of measurement in S103, or may be an instruction from a user via operation portion 24 for ending (or discontinuance). That is, processing in S129 described above is repeated until the prescribed condition is met.

When the prescribed condition for ending is met (YES in S131), CPU 11 sends a control signal to control circuit 14 to drive suction pump 16 via switching valve 17 (S133). With this, a pressed state of the sensor portion against the measurement region is released, and a series of measurement processing is ended.

As described above, in the embodiment, CPU 11 controls multiplexer 20 with switching between an operation for a channel scan in S105 and an operation for fixing the channel in S119. In the pulse wave detection device in this embodiment, the channel can be fixed as such because there is a low possibility of deviation of the channel due to movement of a body during pulse wave measurement, since a time for pulse wave measurement is as short as about 30 seconds to 2 minutes.

Next, characteristic variable filter 22 in the embodiment of the present invention will be described using FIGS. 6-8.

FIG. 6 is a diagram for describing a filter switching circuit forming characteristic variable filter 22 in the embodiment. Referring to FIG. 6, characteristic variable filter 22 is formed with filter 221 and filter 222 having different frequency characteristics, and a switching circuit for switching between these filters. FIGS. 7A and 7B respectively show frequency characteristics of filter 221 and filter 222 shown in FIG. 6.

In this embodiment, as an example, it is assumed that a switching frequency “fx” of pressure signals from 40 sensor elements 28 is 20 kHz. Then, a sampling frequency “fs” of the pressure signal from one of the 40 sensor elements 28 becomes 500 Hz.

In the following description of the embodiment, sampling frequency “fs” means a sampling frequency of a single pressure signal.

Referring to FIG. 7A, a cutoff frequency “fcA” of filter 221 is set to 250 kHz, for example, a value not less than switching frequency “fx” (20 kHz). On the other hand, referring to FIG. 7B, a cutoff frequency “fcB” of filter 222 is set to 100 Hz, for example, a value lower than half a value of sampling frequency “fs”, that is, fs/2 (250 Hz). In a condition as described above, cutoff frequency “fcB” of filter 222 preferably satisfies 30 Hz<fcB<250 Hz (=fs/2).

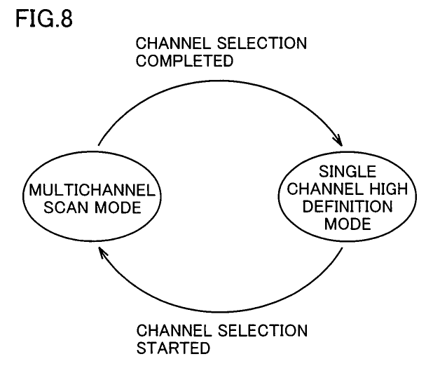

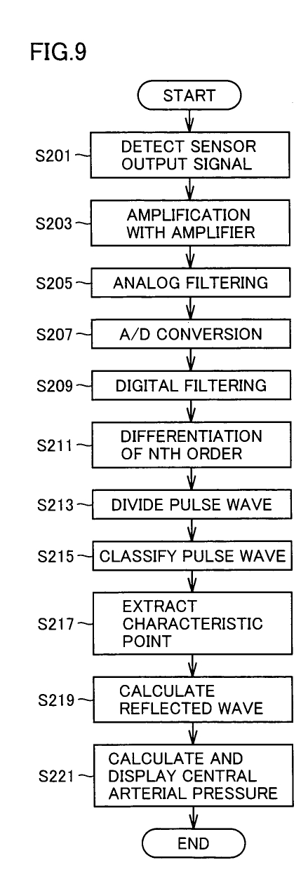

FIG. 8 shows an example of transition of a state (a mode) of characteristic variable filter 22 in the embodiment.

Referring to FIG. 8, when selection of the optimum channel is started with successively switching the pressure signals from the plurality of sensor elements 28 with multiplexer 20, CPU 11 sets characteristic variable filter 22 to a multichannel scan mode. Then, after the selection of the optimum channel, the mode is changed to a single channel high definition mode. In the embodiment, CPU 11 selects filter 221 shown in FIG. 6 to set to the multichannel scan mode. In addition, CPU 11 switches to filter 222 shown in FIG. 6 to set to the single channel high definition mode.

As described above, filter 221 is applied during the selection of the optimum channel since multiplexer 20 is operated to switch pressure signals. Since cutoff frequency “fcA” of filter 221 is set to 250 kHz, which is sufficiently higher than switching frequency “fx” (20 kHz), lack of higher frequency information does not occur during reconstruction of a waveform.

Then, filter 222 is applied after the selection of the optimum channel. Filter 222 functioning as an antialiasing filter can be applied because CPU 11 controls multiplexer 20 to fix to a single channel after the selection of the optimum channel.

Analysis processing of the pressure signal (a sensor signal) obtained from sensor element 28 and processing of calculation and display of a central arterial pressure will now be described referring to FIG. 9. The processing shown in a flow chart of FIG. 9 is also performed by CPU 11 in fixing base unit 7, which accesses ROM 12 to read a program and expands the program on RAM 13 for execution.

Referring to FIG. 9, when a pressure signal is detected in semiconductor pressure sensor 19 having a plurality of sensor elements 28 (S201), semiconductor pressure sensor 19 outputs the pressure signal to amplifier 21 via multiplexer 20. The pressure signal detected in semiconductor pressure sensor 19 is amplified with amplifier 21 to a prescribed frequency (S203), and passed through filter 221 or filter 222 forming characteristic variable filter 22 for analog filtering (S205).

Filter 221 is applied by CPU 11 until the channel is fixed in S119 shown in FIG. 5, and filter 222 is applied after the channel is fixed in S119.

The pressure signal passed through characteristic variable filter 22 is converted into a digital signal in A/D conversion portion 23 (S207), and subject to digital filtering for extracting a frequency in a prescribed range for a purpose of, for example, eliminating a noise (S209). Then, A/D conversion portion 23 transfers the pressure signal in a digital form to CPU 11.

A series of sensor signal analysis processing is ended after processing in S209 until the channel is fixed in S119 described above.

After the channel is fixed in S119, CPU 11 receives the pressure signal from A/D conversion portion 23 and executes the program stored in ROM 12 for differentiation of Nth order of a pulse waveform obtained from the received pressure signal (S211). Then, the pulse waveform is divided based on a result of the differentiation to extract the pulse waveform for one pulse (S213), and the pulse waveform is classified (S215). Then, a prescribed characteristic point is extracted from the pulse waveform classified (S217), and a waveform of a reflected wave of a pulse wave of a peripheral arterial pressure is calculated based on the characteristic point extracted (S219). Thereafter, the sensor signal analysis processing is ended.

Since a form of the pulse waveform can be indicated more clearly using multidimensional (Nth order) differentiation, the characteristic point can also be extracted with high accuracy in the following step of extracting the characteristic point. Then, a second systolic component can also be obtained with high accuracy because the second systolic component (reflected wave) can be obtained from this characteristic point.

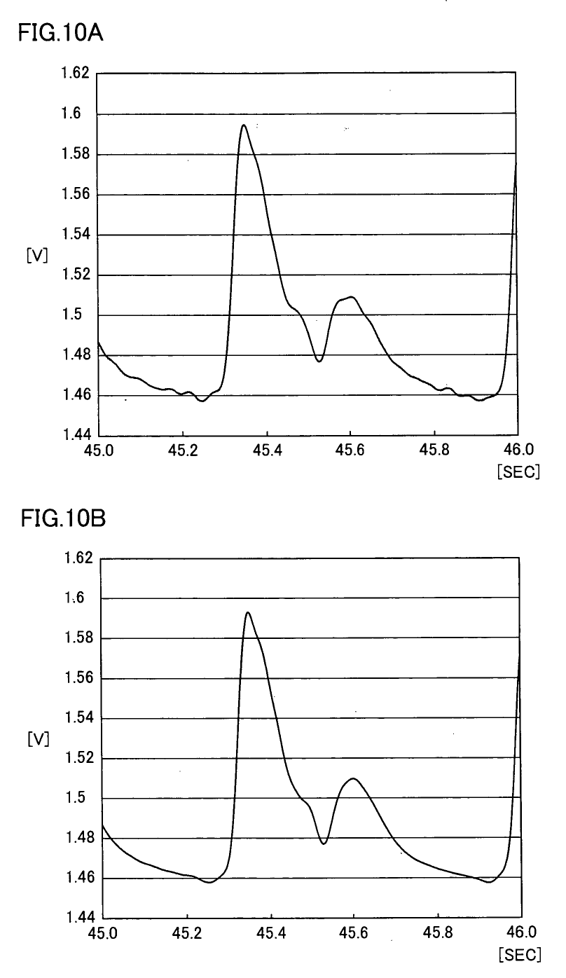

FIG. 10A shows an example of display of the pulse wave for one pulse when the frequency characteristic of characteristic variable filter 22 is set to characteristic “A” in this embodiment. This situation is in the multichannel scan mode, and filter 221 is applied. On the other hand, FIG. 10B shows an example of display of the pulse wave for one pulse when the frequency characteristic of characteristic variable filter 22 is set to characteristic “B”. This situation is in the single channel high definition mode, and filter 222 is applied. In each of FIGS. 10A and 10B, an axis of ordinates indicates a sensor output voltage (V) showing an amplitude level of the pulse wave, and an axis of abscissas indicates a time (sec).

Referring to FIG. 10A, a small amplitude is seen in waveform data in a time period having a small variation in a voltage (for example, near 45.2 seconds and near 45.8 seconds), which shows that a noise is included. Referring to FIG. 10B, on the other hand, a smooth curve is shown even for the waveform data in the time period having a small variation in a voltage (for example, near 45.2 seconds and near 45.8 seconds), which shows that the noise is eliminated.

Therefore, since the pulse waveform for one pulse extracted in S213 of FIG. 9 is highly accurate, the waveform of the reflected wave can be calculated with high accuracy in S219 from the pulse waveform for one pulse, and thus estimation of a systolic blood pressure of a central artery using the reflected wave component can be performed with high accuracy.

It is to be noted that, an AI (Augmentation Index) indicating a characteristic of the pulse waveform extracted in S213 may be obtained and indicated. In this situation, the AI may be indicated in association with the estimated central arterial pressure so as to show a variation with time. The AI is a known index as a blood pressure, which indicates a characteristic amount reflecting intensity of reflection of a pulse wave (a reflection phenomenon of a pulse wave which represents acceptability of an outgoing blood flow) mainly corresponding to arteriosclerosis of a central blood vessel. The AI is recognized as an effective index to find especially a circulatory disease at an early stage, and is known to behave differently from the blood pressure.

In this embodiment, an index such as ΔTp may be calculated and indicated, which is known as a characteristic amount of a pulse wave.

<Modified Example of Pulse Wave Detection Device>

A modified example of a construction of characteristic variable filter 22 in the pulse wave detection device will now be described. The other constructions of the pulse wave detection device are similar to those described above.

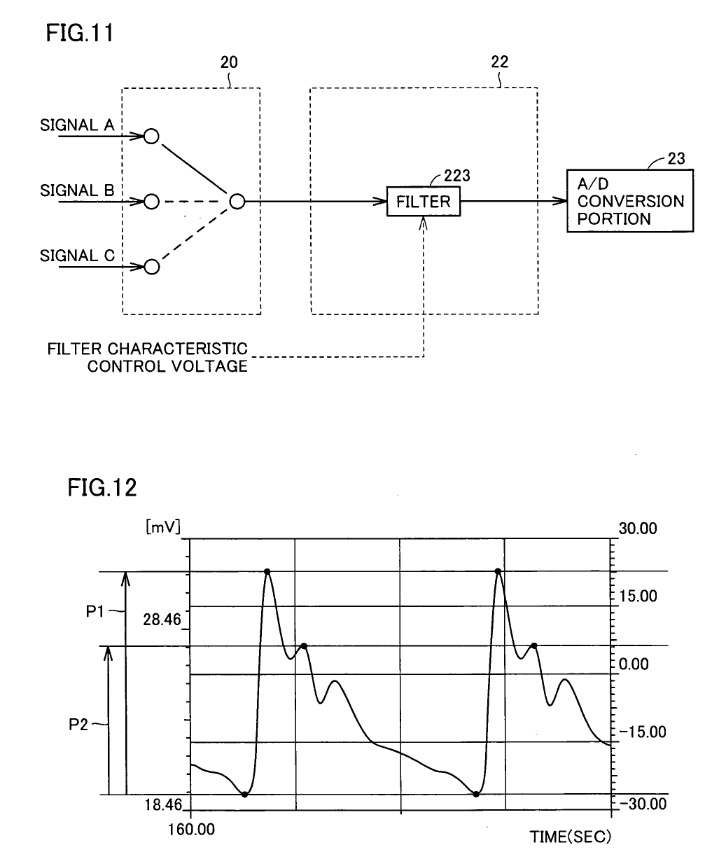

FIG. 11 shows the modified example of the construction of characteristic variable filter 22.

Referring to FIG. 11, in the modified example, a filter 223 having a variable characteristic is used in place of a plurality of analog filters having different characteristics. Filter 223 is formed with a variable capacitance diode. CPU 11 applies a voltage to filter 223 from a control circuit which is not shown. With this, a cutoff frequency in filter 223 can be varied.

In this modified example, in S107 in FIG. 5, CPU 11 applies a control voltage so that a cutoff frequency “fcC” in filter 223 becomes a value of at least switching frequency “fx”. In this situation, it is assumed that cutoff frequency “fcC” is set to, for example, 250 kHz.

In addition, in S121 in FIG. 5, CPU 11 applies a control voltage to vary cutoff frequency “fcC” to, for example, 100 Hz so that filter 223 functions as an antialiasing filter.

With the construction as the modified example, size reduction can be attained because provision of a plurality of analog filters having different characteristics is not required.

It is to be noted that, though the variable capacitance diode is used in the modified example to vary a cutoff frequency component, an element is not limited to this as long as it can vary the cutoff frequency component.

According to the embodiment of the present invention as described above, since multiplexer 20 and characteristic variable filter 22 are dynamically controlled, a channel can be selected appropriately. Therefore, pulse wave data with high accuracy can be obtained.

With this, the pulse wave data for one pulse can be utilized for various analyses. As an example, a variation in movement of a heart after an administration of a medicine to a subject can be detected in real time on a pulse-by-pulse basis.

In addition, a time required for pulse wave measurement can be decreased since a pulse wave analysis for each pulse is enabled.

It is to be noted that, though one sensor element 28 is adopted as the optimum channel in this embodiment, two or more sensor elements may be adopted provided that a number thereof is smaller than a total number of sensor elements 28.

<Procedure for Central Arterial Pressure Calculation>

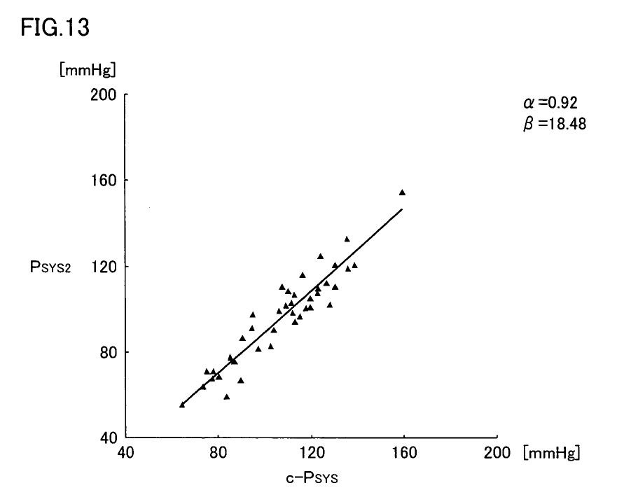

A procedure of central arterial pressure calculation (estimation) in S221 of FIG. 9 will now be described referring to FIGS. 12 and 13.

A systolic blood pressure of a central artery is obtained here as a central arterial pressure. A systolic blood pressure of a central artery can be calculated and estimated with a linear transformation using a prescribed arithmetic expression using a second systolic component generated from a reflected wave (a second systolic component detected in a pressure pulse wave of a peripheral artery) and a systolic blood pressure and a diastolic blood pressure obtained in S104 using electronic sphygmomanometer 30. Since simple arithmetic with a linear transformation is applied for estimation, CPU 11 does not need to have high computing throughput, and estimation can be completed in a short time.

In FIG. 12, based on the waveform of the pressure pulse wave including the reflected wave of the peripheral artery detected in S217 and S219 in FIG. 9, P1 represents a difference between a minimum level and a peak level of an amplitude of the pressure pulse wave, P2 represents a difference between a minimum level and a peak level of an amplitude of a second systolic component of the pressure pulse wave generated from the reflected wave, P_{SYS} (mmHg) represents a peripheral arterial pressure measured in the same period with electronic sphygmomanometer 30, that is, a systolic blood pressure, and P_{DIA} (mmHg) represents a diastolic blood pressure. In this situation, a pressure P_{SYS2} (mmHg) of the second systolic component of the peripheral artery is derived from the following expression 1.
P_{SYS2}=P2/P1×(P_{SYS}−P_{DIA})+P_{DIA} (Expression 1)

When pressure P_{SYS2} of the second systolic component in the peripheral artery is obtained, a systolic blood pressure of a central artery “c-P_{SYS}” is then derived from an expression 2.
c-P_{SYS}=α×P_{SYS2}+β (Expression 2)

Variables α and β in expression 2 can be obtained from a slope and an intercept of an expression of a straight line shown in FIG. 13. That is, α=0.92 and β=18.48. In a graph of FIG. 13, an axis of ordinates indicates pressure P_{SYS2} (mmHg) and an axis of abscissas indicates the systolic blood pressure of a central artery “c-P_{SYS}” (mmHg) to show a relationship between the systolic blood pressure of the central artery “c-P_{SYS}” and pressure P_{SYS2} of the second systolic component of the peripheral artery. Since they have a close relationship with each other, the straight line as shown can be obtained. Values plotted on the graph were obtained beforehand from actual measurement for a subject (a patient) in a normal condition.

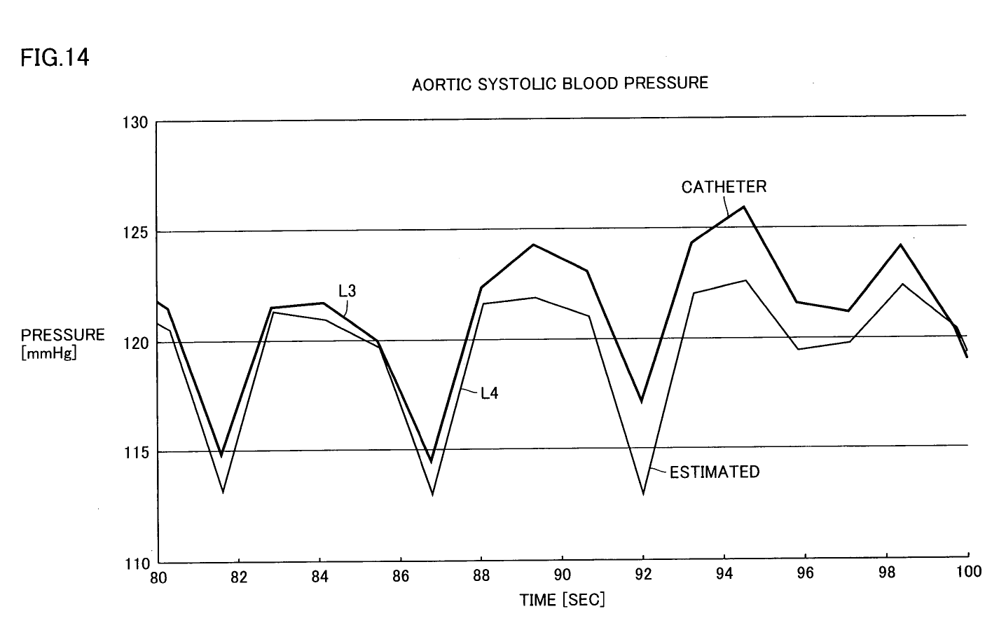

As described above, the systolic blood pressure of the central artery “c-P_{SYS}” can be calculated and estimated by the linear transformation using expressions 1 and 2. The estimated systolic blood pressure of the central artery “c-P_{SYS}” is displayed on display portion 25 in S221 of FIG. 9. An example of a manner of such display is a trend graph L4 shown in FIG. 14.

In FIG. 14, an axis of ordinates indicates a blood pressure (mmHg) and an axis of abscissas indicates a lapse of time (sec) to show trend graphs L3 and L4. FIG. 14 shows variations with time as trend graphs L3 and L4, each obtained by continuously linking a value of a systolic blood pressure for every one heart beat in time sequence, which value was obtained with each of invasive measurement with a catheter and non-invasive measurement with the calculation (estimation) in S221 concurrently performed as to a systolic blood pressure of a central artery of the same subject in an experiment.

As is obvious from a comparison between trend graphs L3 and L4, a trend in a variation in the systolic blood pressure of the central artery estimated in this embodiment nearly corresponds to a trend in a variation in an actually measured blood pressure value.

<Display of Trend of Central Arterial Pressure>

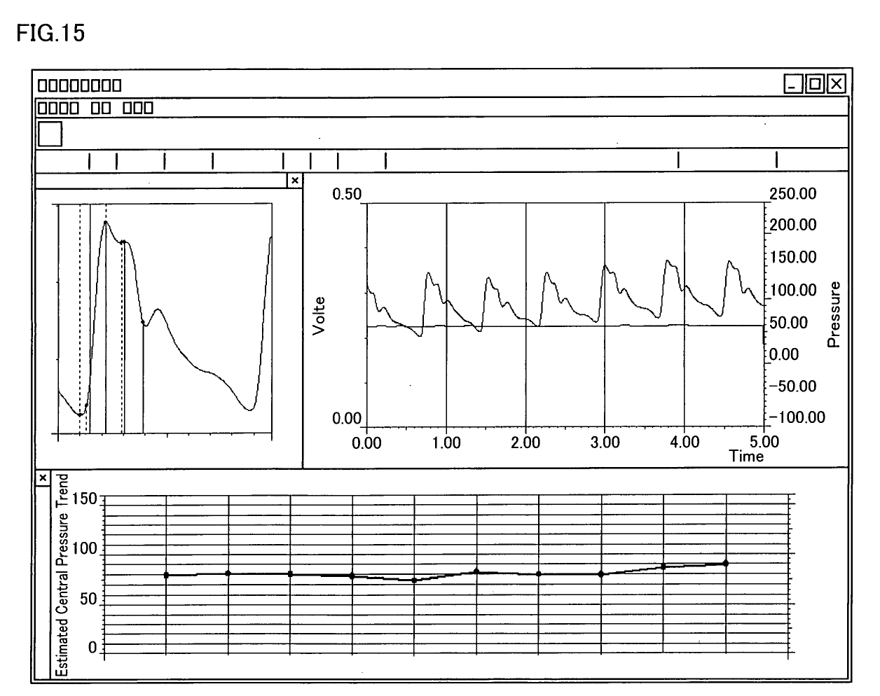

FIG. 15 shows an example of a trend of a central arterial pressure displayed on a screen of display portion 25 in S221. A lower area of the screen in FIG. 15 shows a trend graph obtained by plotting of an estimated central arterial pressure for each heart beat in time sequence. An upper-right area shows a variation with time in a waveform of a pressure pulse wave detected from a peripheral artery, and an upper-left area shows an enlarged state of one pressure pulse wave selected from the upper-right area.

In an administration of a drug such as nitroglycerine, a doctor can recognize a variation with time in a systolic blood pressure of a central artery before and after a drug administration by observing the trend graph in the lower area of the screen shown in FIG. 15, as described with FIG. 20. As a result, an effect of the drug administration such as a decrease in the systolic blood pressure in the central artery can be ensured.

In FIG. 15, an axis of abscissas of the trend graph indicates a number of pulses (heart beats) and an axis of ordinates indicates a blood pressure (mmHg). Though a time axis of the trend graph is calibrated in constant time periods, it may be calibrated as follows. That is, a portion of the graph may be displayed in a successively compressed form in a direction of the time axis after a lapse of a prescribed time from measurement. With this, a trend throughout a relatively long period in the past can be observed in a compressed portion, and a trend of recent time including the present time can be ensured in detail in the other portion which is not compressed. In addition, the axis of abscissas in FIG. 15 may indicate a time (sec) rather than the number of pulses.

<Another Example of Display>

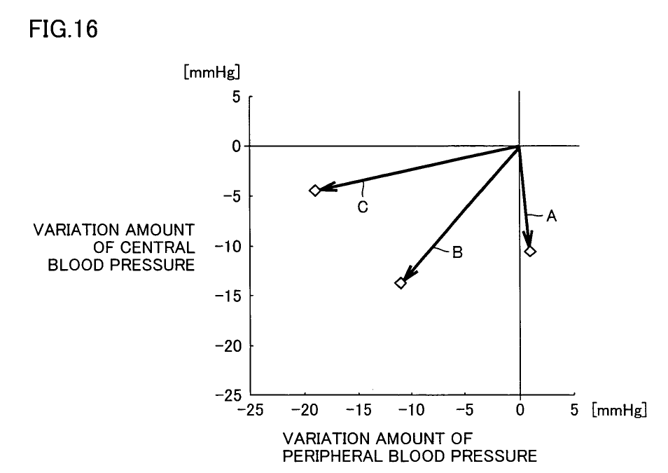

A screen display shown in FIG. 16 may also be adopted. In FIG. 16, an axis of abscissas indicates a variation amount (mmHg) of a blood pressure (a systolic blood pressure) of a peripheral artery measured with electronic sphygmomanometer 30, and an axis of ordinates indicates a variation amount (mmHg) of a blood pressure (a systolic blood pressure) of a central artery calculated (estimated) in S221 of FIG. 9 to show a relationship between them on a two-dimensional graph. Each of arrows A, B and C represents a relationship of variation amounts (differences) between a blood pressure value immediately before a drug administration, which is represented as a point with both variation amounts of 0 (an origin point) on the graph, and a blood pressure value obtained in an arbitrary timing after the drug administration.

When a relationship of variation amounts as an arrow A is detected, a situation is indicated in which only a systolic blood pressure of a central artery drops (a variation in a blood pressure cannot be observed in a peripheral artery). When a relationship of variation amounts as an arrow B is detected, it is recognized that systolic blood pressures drop in both of the central and peripheral arteries. When a relationship of variation amounts as an arrow C is detected, it is recognized that only a systolic blood pressure of a peripheral artery drops and a systolic blood pressure of a central artery is not varied.

When myocardial infarction occurs, for example, a flow in the central artery must be increased for alleviation. Since an internal pressure of the central artery must be decreased to increase the flow, a doctor administrates to a patient a drug such as nitroglycerine which acts as a vasodilator. In this situation, when a time of occurrence of myocardial infarction is set as the origin point of the graph in FIG. 16, effectiveness of an administration of the drug is ensured if the relationship as arrow A or B is indicated in an arbitrary timing after the administration, while ineffectiveness of the administration of the drug is ensured if the relationship as arrow C is indicated.

<Modified Example>

Though a maximum blood pressure and a minimum blood pressure (a systolic blood pressure and a diastolic blood pressure) of a peripheral artery are measured beforehand using electronic sphygmomanometer 30 in S104 in the flow chart of FIG. 5, they may be measured beforehand in S221.

Although the present invention has been described and illustrated in detail, it is clearly understood that the same is by way of illustration and example only and is not to be taken by way of limitation, the spirit and scope of the present invention being limited only by the terms of the appended claims.

1. A central blood pressure estimation device, comprising:
a pulse wave detection portion brought into contact with a surface of a living body for detecting a pulse wave of an artery directly below;
a portion for obtaining a second systolic component in said pulse wave detected by said pulse wave detection portion;
an estimation portion for estimating a variation with time in a systolic blood pressure of a central artery of said living body using said second systolic component obtained with said portion for obtaining a second systolic component; and
a display portion for displaying the variation with time in said systolic blood pressure estimated by said estimation portion.

2. The central blood pressure estimation device according to claim 1, wherein
said estimation portion estimates said systolic blood pressure of said central artery of said living body using a blood pressure of said living body measured beforehand and said second systolic component obtained with said portion for obtaining a second systolic component.

3. The central blood pressure estimation device according to claim 2, wherein
the blood pressure of said living body measured beforehand is a blood pressure of a peripheral artery of the living body.

4. The central blood pressure estimation device according to claim 1, wherein
a linear transformation is used in estimation of said estimation portion.

5. The central blood pressure estimation device according to claim 1, wherein
said display portion successively displays said systolic blood pressure estimated by said estimation portion in time sequence.

6. The central blood pressure estimation device according to claim 5, wherein
said systolic blood pressure for each heart beat is successively displayed.

7. The central blood pressure estimation device according to claim 1, wherein
said display portion displays said systolic blood pressure or a variation thereof with time associated with another index relating to a blood pressure.

8. The central blood pressure estimation device according to claim 7, wherein
said index is a blood pressure of a peripheral artery of said living body or a variation thereof with time.

9. The central blood pressure estimation device according to claim 8, wherein
said display portion displays said systolic blood pressure or a variation thereof with time and the blood pressure of the peripheral artery of said living body or a variation thereof with time in comparison with each other on a two-dimensional graph.

10. A central blood pressure estimation device, comprising:
a pulse wave detection portion brought into contact with a surface of a living body for detecting a pulse wave of an artery directly below;
a portion for obtaining a second systolic component in said pulse wave detected by said pulse wave detection portion;
a calculation portion for calculating a variation with time in the second systolic component obtained with said portion for obtaining a second systolic component; and
a display portion for displaying the variation with time in the second systolic component calculated by said calculation portion.

11. A method for estimating a central blood pressure, comprising the steps of:
detecting a pulse wave using a sensor brought into contact with a surface of a living body to detect a pulse wave of an artery directly below;
obtaining a second systolic component in said pulse wave detected in said step of detecting a pulse wave;
estimating a systolic blood pressure of a central artery of said living body using said second systolic component obtained in said step of obtaining a second systolic component; and
displaying a variation with time in said systolic blood pressure estimated in said step of estimating.

12. A method for estimating a central blood pressure, comprising the steps of:
detecting a pulse wave with contact with a surface of a living body to detect a pulse wave of an artery directly below;
obtaining a second systolic component in the pulse wave detected in said step of detecting a pulse wave;
calculating a variation with time in the second systolic component obtained with said step of obtaining a second systolic component; and
displaying the variation with time in the second systolic component calculated in said step of calculating.
